(19) United States
(12) Patent Application Publication
(10) Pub. No.: US 2008/0287781 A1
(43) Pub. Date: Nov. 20, 2008

(54) Registration Methods and Apparatus

(75) Inventors: Ian Revie, Boroughbridge (GB); Alan Ashby, York (GB); Michal Slomczykowski, Leeds (GB)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DEPUY INTERNATIONAL LIMITED, Leeds (GB)

(21) Appl. No.: 10/598,601

(22) PCT Filed: Mar. 7, 2005
(86) PCT No.: PCT/GB2005/000874

§ 371 Date: Aug. 7, 2008

Related U.S. Application Data
(60) Provisional application No. 60/575,402, filed on Jun. 1, 2004.

(30) Foreign Application Priority Data
Mar. 5, 2004 (GB) 0405011.8

(51) Int. Cl. A61B 5/05

(52) U.S. Cl. 600/426; 382/131

(57) ABSTRACT

A method, system and computer program for generating a registered image of a body part of a patient for use in an image guided surgical procedure is described. The method includes attaching a marker detectable by a tracking system to the body part before carrying out surgical steps. The tracking system has a reference frame and the position of the marker in the reference frame is detected. A first image of the body part is captured using an imaging system. An indication of the position of the first image relative to the reference frame of the tracking system is obtained. The first image is then mapped into registration with the position of the body part. The system includes an imaging system for capturing images of the body part. A tracking system detects a marker and determines the position of the marker in the reference frame of the tracking system. A marker is attachable to the body part and detectable by the tracking system. A computer control system is configured to obtain an indication of the position of the first image relative to the reference frame of the tracking system and to map the first image into registration with the position of the body part.

The present invention relates to methods and apparatus for use in registering images of body parts with body parts, and in particular to methods, systems, apparatus, computer program code and computer program products for producing registered images for use in image guided surgical procedures, and in particular orthopaedic procedures.

Various methods can be used to register images of body parts with the reference frames of tracking systems. However, many of these require the presence of imaging systems within the operating theatre which facilities are not necessarily available.

The present invention therefore relates to being able to provide an image in a format in which the image can be registered or is already automatically registered for use in a surgical procedure.

According to a first aspect of the present invention, there is provided a method for generating a registered image of a body part of a patient for use in a computer aided surgical procedure. The method can comprise attaching a marker detectable by a tracking system to the body part prior to any surgical steps of the surgical procedure. The tracking system has a reference frame. The position of the marker in the reference frame is detected. A first image of the body part or a part of the body part is captured using an imaging system. An indication of the position of the first image relative to the reference frame of the tracking system is obtained. A mapping required to bring the first image into registration with the position of the body part is determined.

In this way, the invention provides an image in a form registerable with the position of the body part and so a registered image can be generated for use in a surgical procedure before any invasive acts associated with the surgical procedure are carried out. Hence the image is pre-registered or automatically registered, or sufficient data exists that the image is in a condition that the image can be brought into registration with the position of a body part when required for use in the computer aided surgical procedure.

The method can further comprise mapping the first image into registration with the position of the body part. Hence in this way a registered image can be generated.

Obtaining an indication of the position of the at least first image relative to the reference frame of the tracking system includes detecting the position in the reference frame of the tracking system of a further marker attached to a part of the imaging system using the tracking system.

The first image can include the marker and at least a part of the body part, wherein the position of the marker is detected when the first image is captured thereby providing the indication.

The method can further comprise displaying the registered image during the image guided surgical procedure. The surgical procedure can be an orthopaedic procedure. The orthopaedic procedure can be carried out on a knee or hip and can include implanting an orthopaedic prosthetic implant or implants.

Attaching the marker can include attaching the marker to the skin of the patient. Attaching the marker can includes implanting the marker in a bone of the patient. Implanting the marker can includes percutaneously implanting the marker.

The marker can be wirelessly detectable by the tracking system, and the marker can be wirelessly detectable at radio frequencies. The marker can be wire based. The marker can be acoustically detectable, e.g. by ultrasound, or electromagnetically detectable, including using infra-red radiation.

The imaging system can be an X-ray system. The X-ray system can be a fluoroscopic X-ray system. The X-ray system can include an x-ray detector. The x-ray detector can have an imaging plane. The x-ray detector can be an x-ray cassette. The x-ray detector can be a digital or an electronic detector which can generate a digital image or digital image signal.

The position of the marker can be detected with the patient standing. The position of the marker can be detected with the patient's weight imparting a load on the body part being imaged.

Obtaining an indication of the position of the at least first image relative to the reference frame of the tracking system can include determining the position of an X-ray detector in the reference frame of the tracking system.

The method can further comprise capturing a second image of the body part using the X-ray system. The second image can be in a second direction different to a first direction in which the first image was captured. A three dimensional image of the body part can be derived from the first and second images. A three dimensional image of the body part in an orientation of the body part corresponding to the orientation of the patient during the surgical procedure can be generated from the first and second images.

Capturing the second image can include moving the patient relative to the X-ray system. Capturing a second image can include moving an X-ray source relative to the patient. The method can further comprises determining the position of the X-ray source in the reference frame of the tracking system when the first and/or second image is captured. Determining the position of the X-ray source can include wirelessly tracking the position of a marker attached to the x-ray source at radio frequencies. The first image can be captured using a first X-ray source. Capturing the second image can include using a second X-ray source at a second position which is different to a first position of the first X-ray source. The or each x-ray source can be an x-ray camera.

The method can further comprising generating a three dimensional image of the body part from the first and second images.

The method can further comprise determining the distance between the body part and an imaging plane of an X-ray detector along a direction perpendicular to the plane of the imaging plan. The distance can be used to compensate the first image captured by the X-ray detector for linear magnification.

The imaging system can be a CT scan or an MR scan system.

The body part of the patient can be located on a patient support part of the imaging system when the first image is captured. The patient support part can be a table. The method can further comprise determining the position of the patient support part or a part of the patient support part in the reference frame of the tracking system. The method can further include determining the position of the patient support part relative to an imaging plane or region of the imaging system.

The method can further comprising mounting a marker detectable by the tracking system on the patient support part.

The body part of the patient can be located on a patient support part of the imaging system when the first image is captured. The method can further comprise determining the position of an imaging plane or imaging region of the scan system relative to the position of the patient support part or a part of the patient support part.

The first image can include the marker or a part of the marker and at least a part of the body part. The position of the marker can be detected when the first image is captured.

The method can further comprise attaching a further marker detectable by the tracking system to a further body part. The further marker can be attached to the further body part prior to any surgical steps of the image guided surgical procedure. The position of the further marker can be detected in the tracking reference frame. The further marker can be attached to a bone different to a bone that the first marker is attached to. The marker and further marker can be attached to body parts associated with a joint. The marker and further marker can be attached to respective bones on either side of a joint.

Yet further markers can be attached to yet further bones. Each marker can identify itself uniquely. Each marker can indicate its position and/or its orientation. Only one marker per bone can be needed in order to determine the position and/or orientation of the body part in the reference frame of the tracking system.

Determining the mapping or mapping the first image into registration with the position of the body part can include using the position of the further marker or markers. Determining the mapping can include identifying a vector in the reference frame of the tracking system. The mapping can be the opposite vector to the identified vector.

According to a second aspect of the invention a system for generating a registered image of a body part of a patient for use in an image guided surgical procedure is provided. The system can include an imaging system for capturing a first image of the body part, a tracking system for detecting a marker and determining the position of the marker in a reference frame of the tracking system, a marker attachable to the body part of the patient and detectable by the tracking system, and a computer control system configured to obtain an indication of the position of the first image relative to the reference frame of the tracking system and to determine how to map the first image into registration with the position of the body part.

Counterpart features to the preferred features and details of the first embodiment of the invention can also be preferred features of the second aspect of the invention, and vice versa.

The marker can be wirelessly detectable by the tracking system. The marker can be wirelessly detectable by the tracking system at radio frequencies. The marker can be percutaneously implantable in a bone of the patient.

The system can further comprise a further marker detectable by the tracking system mounted on a part of the imaging system. The marker can be mounted on an image capturing or detecting part of the imaging system.

The imaging system can be an X-ray imaging system, including an X-ray fluoroscopy imaging system. The X-ray imaging system including an X-ray source and an X-ray detector having an imaging plane. The system can further comprise a second marker detectable by the tracking system mounted on the X-ray detector. The system can further comprise a third marker detectable by the tracking system mounted on the X-ray source.

The X-ray source can be movable relative to the X-ray detector. Preferably the x-ray source can be rotated or pivoted about by at least 90°.

The system can further comprise a fourth marker detectable by the tracking system and a further X-ray source. The fourth marker can be mounted on the further X-ray source.

The computer control system can be further configured to determine a separation between the marker and an imaging plane of the X-ray detector and to use the separation to correct the first image.

The imaging system can be a CT scan or MR scan imaging system.

The system can further comprise a second marker detectable by the tracking system. The imaging system can includes a patient support part. The second marker can be mounted on the patient support part.

The computer control system can be further configured to determine the position of the patient when the first image includes the marker or at least a part of the marker and at least a part of the body part.

The imaging system can include a patient support part. The computer control system can determine the position of an imaging plane or region of the imaging system relative to the patient support part.

The system can further comprise a further marker or markers attachable to a further body part or body parts of the patient and detectable by the tracking system.

According to a third aspect of the invention, there is provided a computer implemented method for generating a registered image of a body part of a patient bearing a marker detectable by a tracking system having a reference frame, the registered image being for use in a computer aided surgical procedure and being generated prior to any surgical steps of the surgical procedure. The method can comprise determining the position of the marker in the reference frame, determining the position of a first image of the body part relative to the reference frame of the tracking system. The first image can have been captured by an imaging system. A mapping which brings the first image into registration with the position of the body part can be determined.

Counterpart features to the preferred features and details of the first and second embodiment of the invention can also be preferred features of the third aspect of the invention, and vice versa.

The mapping can be a vector in the reference frame of the tracking system.

The first image can be registered with the position of the body part in the reference frame of the tracking system. The first image can be registered with the position of the body part in the reference frame of the computer control system. The registered image can be displayed. The registered image can be derived from the captured image. The registered image can be a rendered image showing the body part at a position and/or orientation different to that of the body part when the image was captured.

The first image can include a further body part of the patient bearing a further marker detectable by the tracking system. The method can further comprise determining the position of the further marker in the reference frame.

Determining the position of the first image of the body part can include determining the position of a further marker detectable by the tracking system and attached to a part of the imaging system.

Determining the position of the first image can include determining the position of an imaging plane or region of the imaging system relative to a patient support part of the imaging system.

Determining the position of a first image of the body part relative to the reference frame can include determining the position of an image of at least a part of the marker in the first image.

The method can further comprise displaying the registered image. The registered image can be displayed during a computer aided surgical procedure. The registered image can be displayed during a computer aided surgical procedure and before any invasive steps associated with the surgical procedure have been carried out.

According to a fourth aspect of the invention, there is provided computer program code executable by a data processing device to provide a system according to a preceding aspect of the invention or a method according to at least one of the preceding method aspects of the invention. A computer readable medium bearing computer program code according to the preceding aspect of the invention is also provided.

An embodiment of the invention will now be described, by way of example only, and with reference to the accompanying drawings, in which:

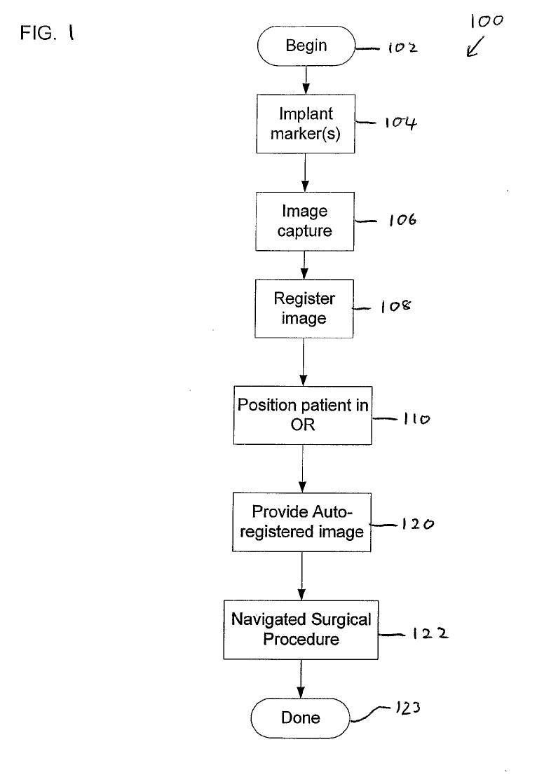

FIG. 1 shows a flow chart illustrating at a high level a patient treatment method in which methods of the invention can be used;

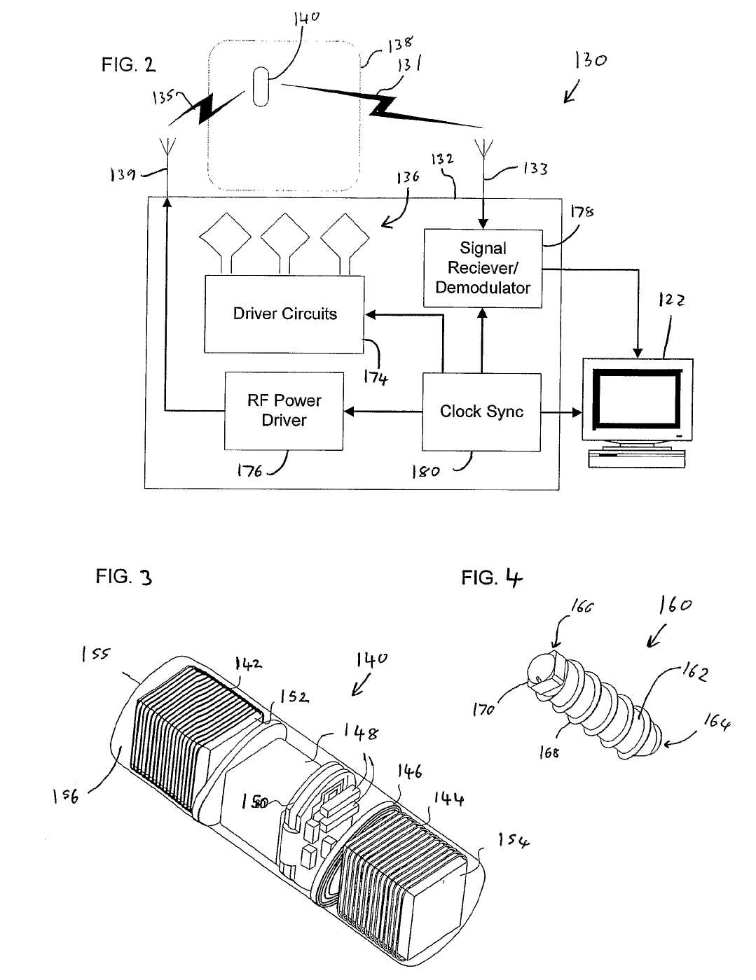

FIG. 2 shows a schematic block diagram of a tracking system and a marker part of a system of the invention;

FIG. 3 shows a perspective view of the marker shown in FIG. 2;

FIG. 4 shows a housing part of an implantable marker part of the system of the invention and useable in methods of the invention;

FIG. 5 shows a schematic diagram of an embodiment of the system according to the invention including the tracking system shown in FIG. 2 and an X-ray imaging system;

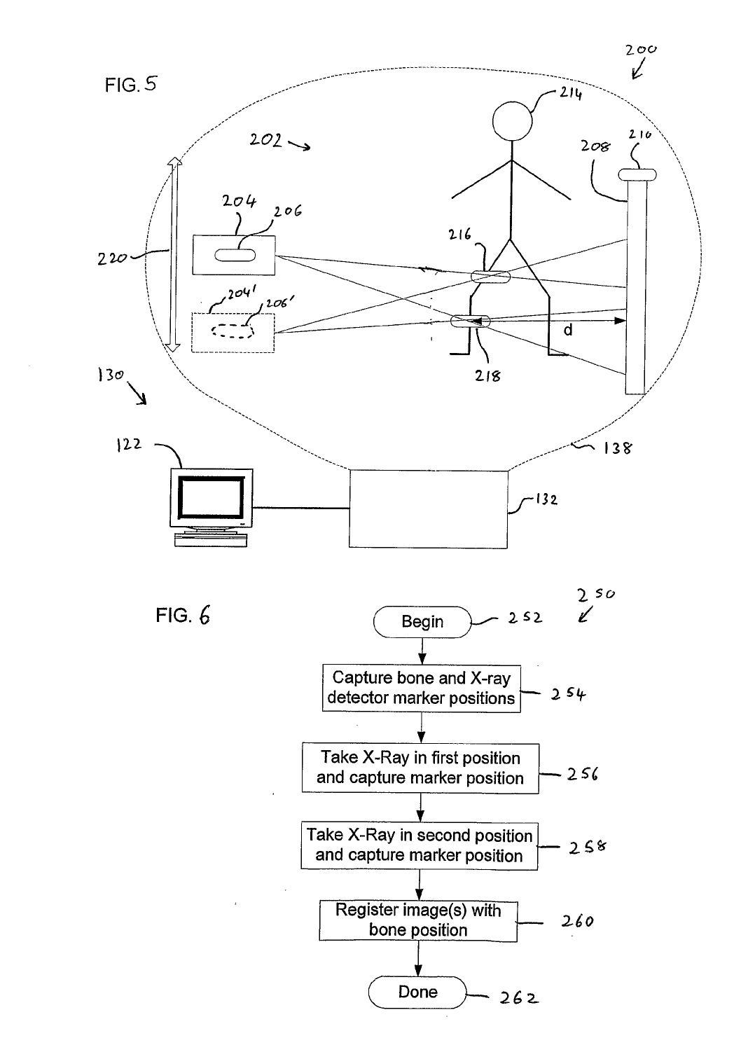

FIG. 6 shows a flow chart illustrating a method of using the system shown in FIG. 5 according to the invention;

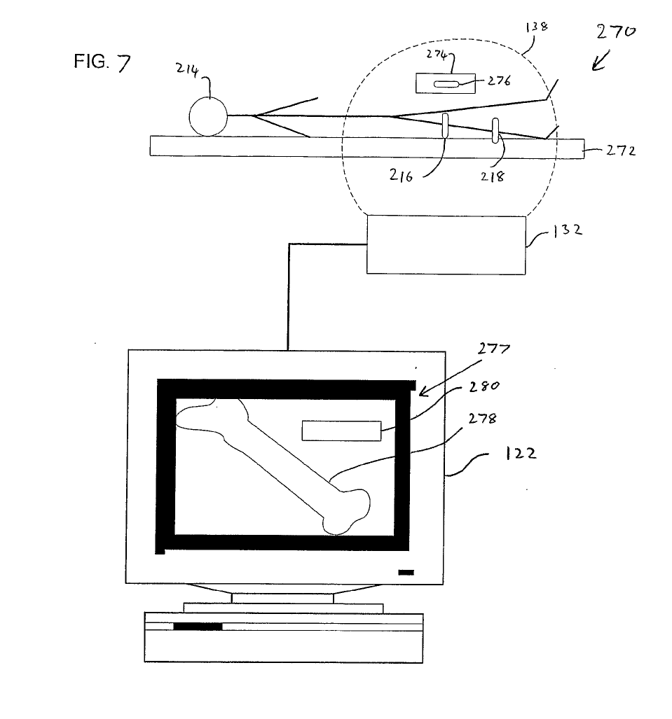

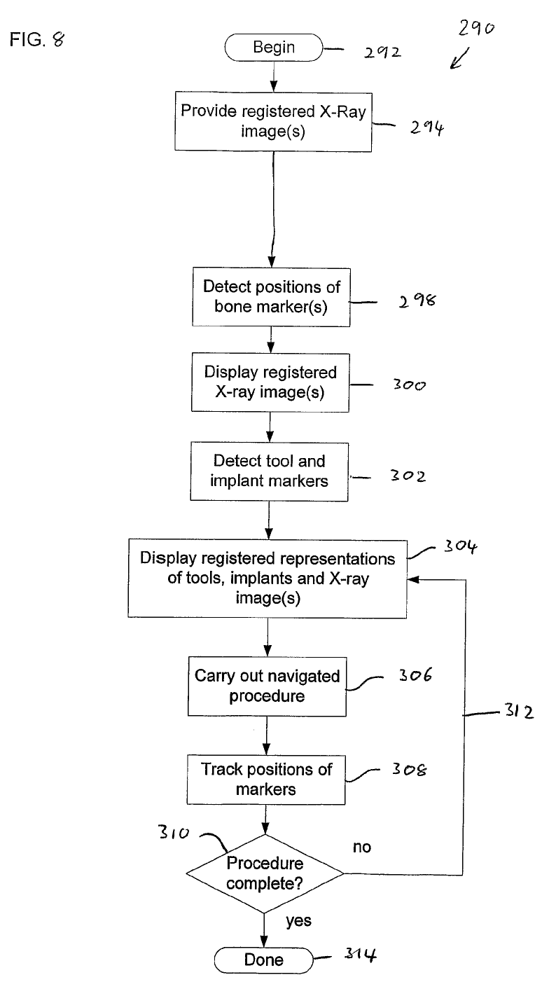

FIG. 7 shows a schematic illustrating of the display of a registered image in an operating theatre as part of an image guided surgical procedure;

FIG. 8 shows a flow chart illustrating a method of providing the registered image as part of an image guided surgical procedure;

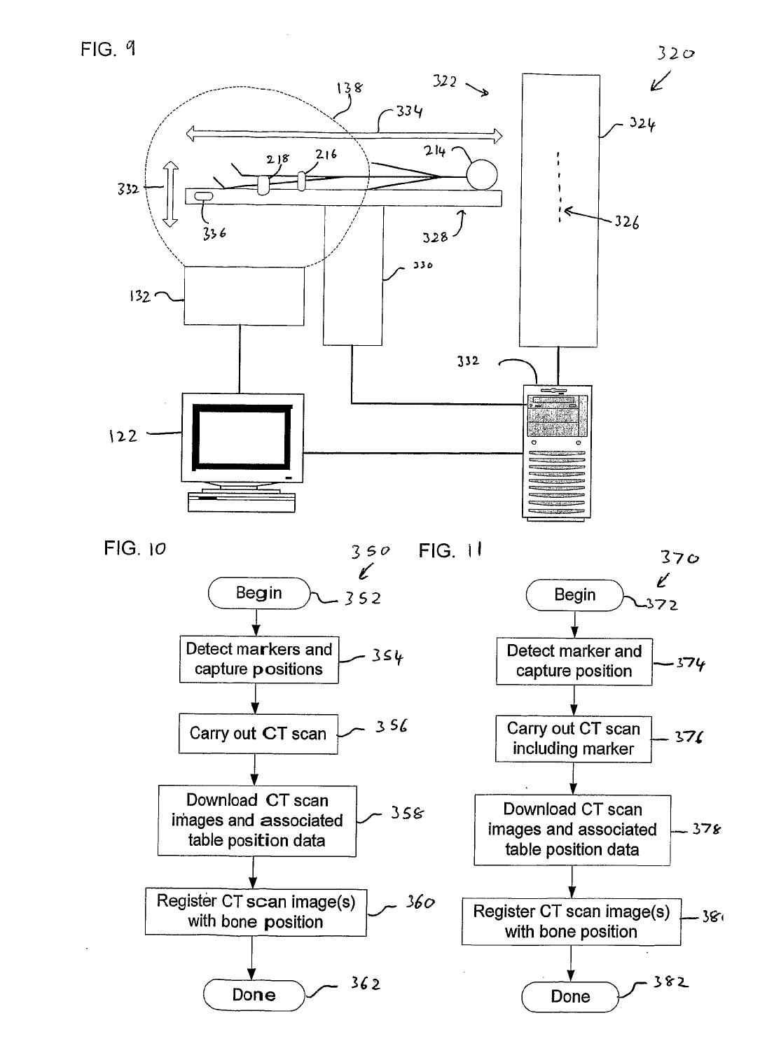

FIG. 9 shows a schematic diagram of a further embodiment of the system according to the invention including the tracking system shown in FIG. 2 and a CT imaging system;

FIG. 10 shows a flow chart illustrating a method of using the system shown in FIG. 9 according to the invention;

FIG. 11 shows a flow chart illustrating a further method of using the system shown in FIG. 9 according to the invention;

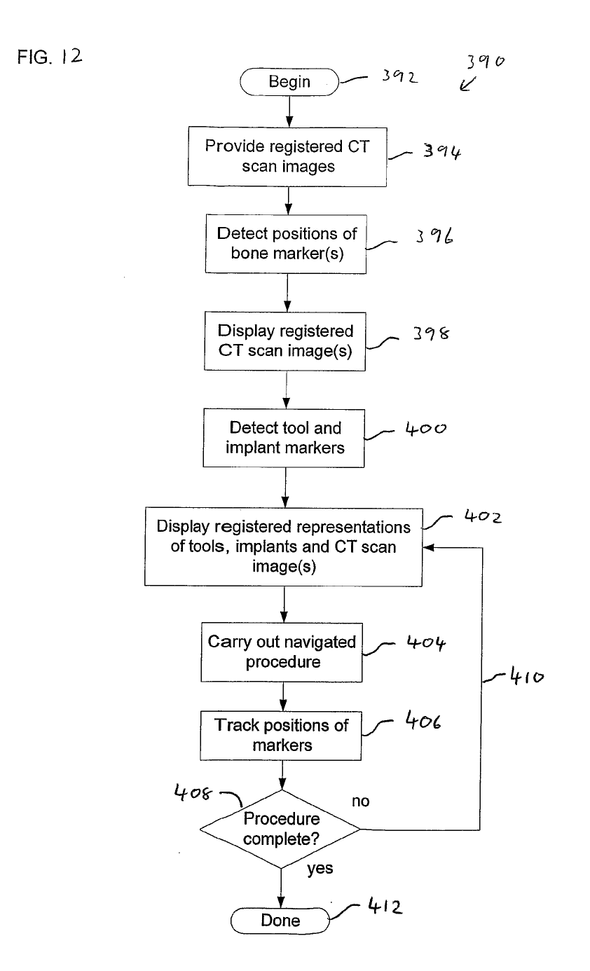

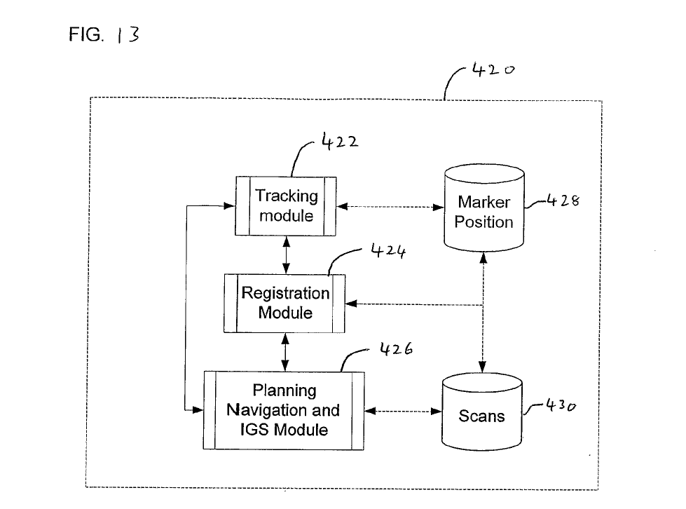

FIG. 12 shows a flow chart illustrating a further method of providing the registered image as part of an image guided surgical procedure;

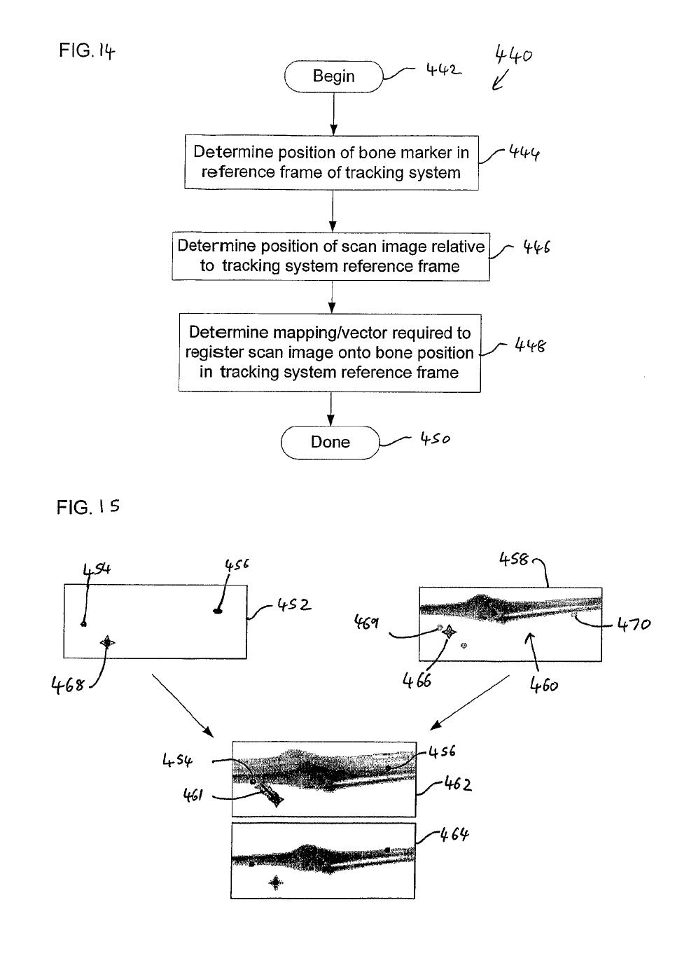

FIG. 13 shows a schematic software architecture of a computer control part of the system of the invention;

FIG. 14 shows a flow chart illustrating a computer implemented method of providing a registered body part image according to the invention;

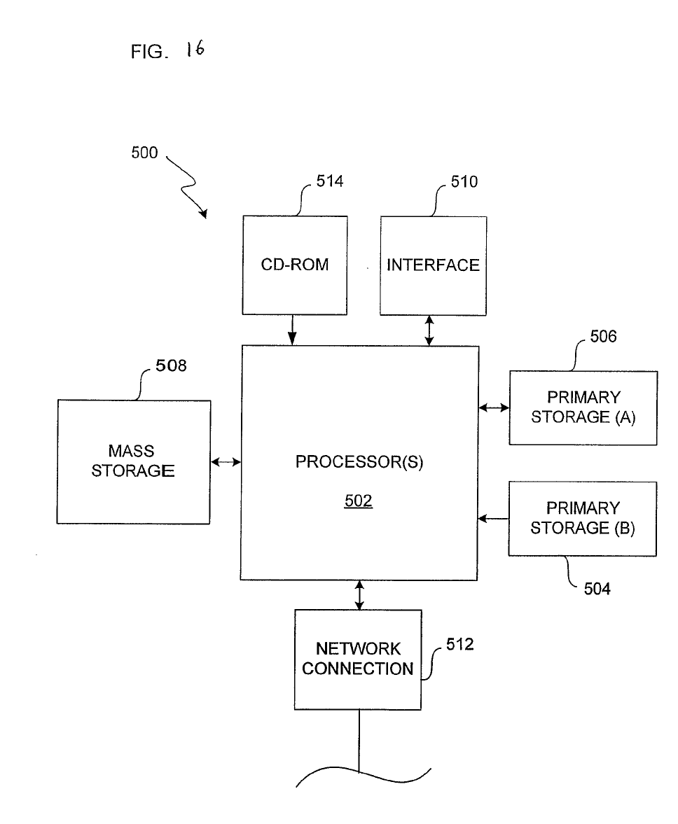

FIG. 15 shows a graphical representation of the registration method illustrated in FIG. 14; and

FIG. 16 shows a schematic block diagram of a computer part of the system of the invention.

Similar items in different Figures share common reference numerals unless indicated otherwise.

With reference to FIG. 1 there is shown a flowchart illustrating, at a high level, a general method 100 in which the registration method of the present invention can be utilised. The present invention allows a registered image to be provided for use in a computer aided surgical procedure, in which images of the patient's body parts are pre-registered, or automatically registered and available for use in the surgical procedure. The method is particularly advantageous as the patient's body parts images are registered without requiring any invasive surgical procedure to be carried out on the patient. This therefore reduces the time of, and complexity of, the computer aided surgical procedure. The image registration method can be carried out as an out patient or clinical procedure some time before the computer aided surgical procedure, e.g. several days or weeks, or immediately prior to the surgical procedure.

The method begins at step 102 and at step 104 markers detectable by a checking system so as to determine the position of the markers are attached to the patient's body part. Various types of markers and associated tracking technology can be used. For example, an acoustic or ultrasound based tracking system can be used. Or alternatively, a wire based tracking system can be used. In other embodiments, various wireless tracking technologies can be used, such as an infrared based tracking technology, which uses passive markers having infrared reflective spheres. A suitable infrared based tracking technology is available from BrainLab GmbH of Germany.

In particularly preferred embodiment of the invention, a wireless radio frequency based tracking technology is used and the markers are implanted within the bones of the patient through the patient's skin. Suitable percutaneously implantable markers and an associated tracking system are described in greater detail below.

At step 104, a marker is implanted in each of the bones for which a registered image is to be provided during the subsequent surgical procedure. The invention will be described below with reference to a total knee replacement procedure involving prosthetic orthopaedic implants, but it will be appreciated that the method is not limited to that surgical procedure nor to implanting orthopaedic prostheses. Rather, the method of the present invention is useable in any computer aided procedure in which registration of image data relating to a body part with the reference frame of a tracking system is desirable.

At step 106, after the markers have been implanted, an image or several images of the body part, e.g. the femur, knee, tibia and tibia are captured using an imaging system. After the body part images have been captured, at step 108, the body part images, or images of the body part derived from the captured images, are brought into registration with the actual position of the body part, e.g. mapping images of the femur, knee, tibia and tibia into registration with the detected position of the femur, the tibia and fibia. It is not necessary to actually generate a registered image at step 108, but merely to carry out the data processing operations required in order to allow a registered image to be generated, either for display, or to be used or otherwise processed by a computer aided surgical system during the surgical procedure.

After the body part position data and image data have been processed to allow a registered image to be provided at step 108, at step 110, the patient can be located at 110 in the operating room in which the surgical procedure is to be carried out. It will be appreciated that pre-operative steps 104, 106, 108 can be carried out some time e.g. days, weeks, months before the subsequent steps of the operation are carried out. Alternatively, the pre-operative steps can be carried out a matter of hours or minutes before the operation itself is carried out. It will also be appreciated that in a suitably equipped operating room, it will be possible to carry out steps 104 to 108 in the operating room in which case step 110 corresponds to making the patient available for surgery and merely indicates the cross over between the pre-operative steps and the beginning of the steps associated with the surgical procedure. Therefore step 110 does not necessarily require the actual physical movement of the patient in some embodiments of the invention. However, it will be appreciated that no invasive surgical steps have been carried out and that invasive surgical steps only begin later on in the method at step 122.

After the patient has been made available for surgery in the operating room at step 110, at step 120, the registered image can be made available to a computer aided surgical system in the operating room and either displayed by the system, used or otherwise processed by the computer aided surgical system so as to be of assistance in the subsequent computer aided surgical procedure. Then at step 122, the computer aided surgical procedure is begun and can include various steps, including planning the surgical procedure or the navigated use of tools, instruments and implants and the provision of images of the body part in image guided steps of the surgical procedure. The above will be referred to herein generally as computer aided, navigated or image guided surgical steps or methods unless the context implies a more specific meaning. However, all of these share the feature of using an image or image data associated with a body part which has been registered with the actual position of the body part so as to assist in the carrying out of a surgical or other treatment step or operation.

The registered image of the body part can be used in step 122 to aid in the planning of the knee replacement surgery, e.g. by assessing the current kinematic performance of the patient's knee and identifying appropriate positions for the femoral and tibial orthopaedic implants. The navigated guidance of instruments to prepare the patient's knee for the implants which can also involve displaying captured images of the patient's bones together with representations of the tools, and the navigated and image guided positioning of the implants in the patient's knee and finally an assessment of the kinematic performance of the patient's knee after the orthopaedic implants have been implanted. The method 100 then ends at step 123 after the surgical procedure has been completed and when the immediate post-operative assessment of the success of the procedure has been carried out.

It will be appreciated that the method of the present invention is not limited to surgical procedures involving orthopaedic prosthetic implants and it can be of benefit in other surgical procedures in which registered images of the patient's bones would be useful, such as other orthopaedic procedures, including, by way of example only, a cruciate knee ligament replacement procedure. The method is also particularly suitable for use in hip, elbow, shoulder and spinal procedures, and especially those including the implantation of prosthetic and other implants in those joints or bone structures.

A suitable marker 140 and associated tracking system 130 for use in the image registration method will briefly be described in greater detail. Aspects of the marker 140 and tracking sub-system 130 are described in greater detail in U.S. patent publication no. US 2003/0120150 A1 (U.S. patent application Ser. No. 10/029,473) which is incorporated herein by reference in its entirety for all purposes.

With reference to FIGS. 2, 3 and 4 there are shown a schematic block diagram of the magnetic tracking system 130, marker, or wireless position sensor, 140, which can be tracked by the tracking system, and a housing 160 for the marker which provides a percutaneously implantable marker. The marker 140 generates and wirelessly transmits a digital signal 131 encoding data items indicative of the marker's identity, its location (x, y and z co-ordinates within the Cartesian reference frame of the tracking system) and orientation (pitch, roll and yaw), in response to an external magnetic field 138 produced by the three field generator coils 136 (also referred to as radiator coils). The location and orientation of the marker will generally be referred to as the markers position.

FIG. 2 illustrates some of the circuitry in a tracking station part 13 of the tracking system 130 which co-operates with computer based controller 122. Field generator coils 136 are driven by driver circuits 174 to generate electromagnetic fields at different, respective sets of frequencies {w₁}, {w₂} and {w₃}. Typically, the sets comprise frequencies in the approximate range of 100 Hz-0 kHz, although higher and lower frequencies may also be used. In one embodiment frequencies in the range of between approximately 15 kHz-0 kHz are used. The sets of frequencies at which the coils radiate are set by computer 122, which serves as the system controller for the tracking system 130. The respective sets of frequencies may all include the same frequencies, or they may include different frequencies. In any case, computer 122 controls circuits 174 according to a known multiplexing pattern, which provides that at any point in time, no more than one field generator coil is radiating at any given frequency. Typically, each driver circuit is controlled to scan cyclically over time through the frequencies in its respective set. Alternatively, each driver circuit may drive a respective one of coils 136 to radiate at multiple frequencies simultaneously.

For the purposes of tracking station 132, coils 136 may be arranged in any convenient position and orientation, so long as they are fixed in respect to some reference frame, and so long as they are non-overlapping, that is, there are no two field generator coils with the exact, identical location and orientation. Typically, for surgical or during patient image capturing applications the coils are located in a triangular arrangement. The coil axes may be parallel, or they may alternatively be inclined. Bar-shaped transmitters or even triangular or square-shaped coils could also be useful for such applications.

It is desirable that coils 136 be positioned away from the surgical or image capturing field, so as not to interfere with the surgeon's freedom of movement or with the patient or a patient support. On the other hand, the coils should be positioned so that the working volume 138 of the tracking system includes the entire area in which the surgeon is operating or in which the patient's marked body parts are on in which any marked parts of the imaging system are located. At the same time, the locations and orientations of coils 136 should be known relative to a given reference frame in order to permit the coordinates of markers 140 to be determined in that reference frame. In practice, coils 136 are mounted on a reference structure part (not shown) of the tracking station 134.

The markers 140 include sensor coils 142, in which electrical currents are induced to flow in response to the magnetic fields produced by field generator coils 136. The sensor coils 142 may be wound on either air cores or cores of magnetic material. Typically, each marker comprises three sensor coils, having mutually orthogonal axes, one of which is conveniently aligned with a principal axis of the housing 160 or of an orthopaedic implant, such as a longitudinal axis. The three coils may be concentrically wound on a single core, or alternatively, the coils may be non-concentrically wound on separate cores, and spaced along the principal axis. The use of non-concentric coils is described, for example, in the PCT Patent Publication WO 96/05768 and in the corresponding U.S. patent application Ser. No. 09/414,875 which are incorporated herein by reference in their entirety for all purposes.

Alternatively, the markers 140 may each comprise only a single sensor coil or two sensor coils. Further alternatively, markers 140 may comprise magnetic position sensors based on sensing elements of other types known in the art, such as Hall effect sensors.

At any instant in time, the currents induced in the sensor coils 142 comprise components at the specific frequencies in sets {w₁}, {w₂} and {w₃} generated by field generator coils 136. The respective amplitudes of these currents (or alternatively, of time-varying voltages that may be measured across the sensor coils) are dependent on the location and orientation of the marker relative to the locations and orientations of the field generator coils. In response to the induced currents or voltages, signal processing and transmitter circuitry in each marker generate and transmit signals 131 that are indicative of the location and orientation of the sensor. These signals are received by receiving antenna 133, which is coupled to computer 122 via signal receiver and demodulation circuitry 178. The computer processes the received signals, together with a representation of the signals used to drive field generator coils 136, in order to calculate location and orientation coordinates of the implantable marker. The coordinates are processed and stored by the computer 122 as will be described in greater detail below.

Although in FIG. 2 tracking system 130 is shown as comprising three field generator coils 136, in other embodiments, different numbers, types and configurations of field generators and sensors may used. A fixed frame of reference may be established, for example, using only two non-overlapping field generator coils to generate distinguishable magnetic fields. Two non-parallel sensor coils may be used to measure the magnetic field flux due to the field generator coils, in order to determine six location and orientation coordinates (X, Y, Z directions and pitch, yaw and roll orientations) of the sensor. Using three field generator coils and three sensor coils, however, tends to improve the accuracy and reliability of the position measurement.

Alternatively, if only a single sensor coil is used, computer 122 can still determine five position and orientation coordinates (X, Y, Z directions and pitch and yaw orientations). Specific features and functions of a single coil system (also referred to as a single axis system) are described in U.S. Pat. No. 6,484,118, whose disclosure is incorporated herein by reference.

When a metal or other magnetically-responsive article is brought into the vicinity of an object being tracked, the magnetic fields in this vicinity are distorted. There can be a substantial amount of conductive and permeable material in a surgical or imaging environment, including basic and ancillary equipment (operating tables, carts, movable lamps, etc.), invasive surgery apparatus (scalpels, scissors, etc.), and parts of the imaging system, such as X-ray sources and detectors and parts of a CT scanner and patient table. The magnetic fields produced by field generator coils 136 may generate eddy currents in such articles, and the eddy currents then cause a parasitic magnetic field to be radiated. Such parasitic fields and other types of distortion can lead to errors in determining the position of the object being tracked.

In order to alleviate this problem, the elements of the tracking station 132 and other articles used in the vicinity of the tracking system 130 are typically made of non-metallic materials when possible, or of metallic materials with low permeability and conductivity. In addition, computer 122 may be programmed to detect and compensate for the effects of metal objects in the vicinity of the monitoring station. Exemplary methods for such detection and compensation are described in U.S. Pat. Nos. 6,147,480 and 6,373,240, as well as in U.S. patent application Ser. Nos. 10/448,289 filed May 29, 2003 and 10/632,217 filed Jul. 31, 2003, all of whose disclosures are incorporated herein by reference.

Marker 140 in this embodiment comprises three sets of coils: sensor coils 142, power coils 144, and a communication coil 146. Alternatively, the functions of the power and communication coils may be combined, as described in U.S. patent application Ser. No. 10/029,473. Coils 142, 144 and 146 are coupled to electronic processing circuitry 148, which is mounted on a suitable substrate 150, such as a flexible printed circuit board (PCB). Details of the construction and operation of circuitry 148 are described in U.S. patent application Ser. No. 10/029,473 and in U.S. patent application Ser. No. 10/706,298 which are incorporated herein by reference in their entirety for all purposes.

Although for simplicity, FIG. 3 shows only a single sensor coil 142 and a single power coil 144, in practice sensor 140 typically comprises multiple coils of each type, such as three sensor coils and three power coils. The sensor coils are wound together, in mutually-orthogonal directions, on a sensor core 152, while the power coils are wound together, in mutually-orthogonal directions, on a power core 154. Alternatively, the sensor and power coils may be overlapped on the same core, as described, for example in U.S. patent application Ser. No. 10/754,751 filed Jan. 9, 2004, whose disclosure is incorporated herein by reference. It is generally desirable to separate the coils one from another by means of a dielectric layer (or by interleaving the power and sensor coils when a common core is used for both) in order to reduce parasitic capacitance between the coils.

In operation, power coils 144 serve as a power source for sensor 140. The power coils receive energy by inductive coupling from external driving antenna 139 attached to RF power driving circuitry 174. Typically, the driving antenna radiates an intense electromagnetic field at a relatively high radio frequency (RF), such as in the range of 13.5 MHz. The driving field causes currents to flow in coils 144, which are rectified in order to power circuitry 148. Meanwhile, field generator coils 136 induce time-varying signal voltages to develop across sensor coils 142, as described above. Circuitry 148 senses the signal voltages, and generates output signals in response thereto. The output signals may be either analog or digital in form. Circuitry 148 drives communication coil 146 to transmit the output signals to receiving antenna 133 outside the patient's body. Typically, the output signals are transmitted at still higher radio frequencies, such as frequencies in the rage of 43 MHz or 915 MHz, using a frequency-modulation scheme, for example. Additionally or alternatively, coil 146 may be used to receive control signals, such as a clock signal, from a transmitting antenna (not shown) outside the patient's body.

As explained above, an RF power driver 176 is provided, which drives antenna 139 to emit power signal 135, preferably in the 2-10 MHz range. The power signal causes a current to flow in power coil 144, which is rectified by circuitry 148 and used to power the markers internal circuits. Meanwhile, the electromagnetic fields produced by field generator coils 136 cause currents to flow in sensor coil 142. This current has frequency components at the same frequencies as the driving currents flowing through the generator coils 136. The current components are proportional to the strengths of the components of the respective magnetic fields produced by the generator coils in a direction parallel to the sensor coil axes. Thus, the amplitudes of the currents indicate the position and orientation of coils 142 relative to fixed generator coils 136.

Circuitry 148 measures the currents flowing in sensor coils 142 at the different field frequencies. It encodes this measurement in a high-frequency signal, which it then transmits back via antenna 146 to antenna 133. Circuitry 148 comprises a sampling circuit and analog/digital (A/D) converter, which digitizes the amplitude of the current flowing in sensor coils 142. In this case, circuitry 148 generates a digitally-modulated signal, and RF-modulates the signal for transmission by antenna 146. Any suitable method of digital encoding and modulation may be used for this purpose. Circuitry 148 also stores a unique identifier for each marker and similarly generates a digitally-modulated signal, and RF-modulates the signal 131 for transmission by antenna 146. Other methods of signal processing and modulation will be apparent to those skilled in the art.

The digitally-modulated signal transmitted by antenna 146 is picked up by receiver 178, coupled to antenna 133. The receiver demodulates the signal to generate a suitable input to signal processing circuits which can be separate to, or integrated in, the computer system 122. Typically, receiver 178 amplifies, filters and digitizes the signals from marker 140. The digitized signals are received and used by the computer 122 to compute the location and orientation of marker 140. General-purpose computer 122 is programmed and equipped with appropriate input circuitry for processing the signals from receiver 178.

Preferably, a clock synchronization circuit 180, is provided which is used to synchronize driver circuits 174 and RF power driver 176. The RF power driver can operate at a frequency that is an integer multiple of the driving frequencies of field generators 136. Circuitry 148 can then use the RF signal received by power coil 144 not only as its power source, but also as a frequency reference. Using this reference, circuitry 148 is able to apply phase-sensitive processing to the current signals generated by sensor coils 142, to detect the sensor coil currents in phase with the driving fields generated by coils 136. Receiver 178 can apply phase-sensitive processing methods, as are known in the art, in a similar manner, using the input from clock synchronization circuit 180. Such phase-sensitive detection methods enable marker 140 to achieve an enhanced signal/noise (S/N) ratio, despite the low amplitude of the current signals in sensor coils 142.

Although certain frequency ranges are cited above by way of example, those skilled in the art will appreciate that other frequency ranges may be used for the same purposes.

Circuitry 148 also stores a unique identifier for marker 140 and the unique identifier is also transmitted to the tracking system 130, so that the tracking system can determine the identity of the marker from which positional data is being received. Hence the tracking system can discriminate between different markers when multiple markers are present in the working volume 138 of the tracking station.

An advantage of using wireless markers, such as marker 140, without an on-board power source, is that the markers can be inserted in and then left inside the patient's body for later reference.

As illustrated in FIG. 3, marker 140 can be hermetically sealed by encapsulation 155 in a sealant or encapsulant 156. Preferably the sealant provides any, some or all of the following shielding properties: mechanical shock isolation; electromagnetic isolation; biocompatibility shielding. The sealant can also help to bond the electronic components of the marker together. Suitable sealants, or encapsulants, include USP Class 6 epoxies, such as that sold under the trade name Parylene. Other suitable sealants include epoxy resins, silicon rubbers and polyurethane glues. The marker can be encapsulated by dipping the marker in the sealant in a liquid state and then leaving the sealant to set or cure.

Marker 140 can be attached to orthopaedic prosthetic implants so as to allow the position of the orthopaedic implants to be tracked during a navigated or image guided surgical procedure. A housing can be provided around the encapsulant 156 and then the housed marker can be secured to the orthopaedic implant.

FIG. 4 shows a housing 160 for marker 140 which can be used to provide an implantable marker which can be implanted directly in the bone of the patient. In a preferred embodiment, the implantable marker is configured to be “injectable” through the skin of the patient without requiring an ancillary incision or other preliminary surgical procedure. Housing 160 has a generally cylindrical body 162 having a tapered distal end 164 and a screw thread 168 extending along the longitudinal axis of the housing. A connector 170 in the form of a generally square shaped formation is provided at the proximal end 166 for releasably engaging with an insertion instrument so as to impart rotational drive to the housing so that the implantable marker can be screwed into the patient's bone. In one embodiment, the housing has a sharp, skin piercing tip at the distal end with a self-tapping screw thread thereon. The implantable marker can then be percutaneously implanted into the bone by pushing the marker through the skin, optionally using a guide instrument having a guide tube with a guide channel passing there along, and then screwing the implantable marker into the bone.

In another embodiment, no screw thread 168 is provided and the outer surface of the housing is substantially smooth and the distal end has a sharp bone penetrating tip, such as a trochanter tip. In this embodiment, the implantable marker can be percutaneously implanted by driving the implantable marker through the skin and pushing the implantable marker into the bone. While the screw thread provides a bone anchor for some embodiments, in this embodiment, barbs, ribs or other surface features can provide a bone anchor. Alternatively, a bone anchor can be provided by treating the outer surface of the marker housing to encourage, facilitate or otherwise promote bone on growth. For example the outer surface of the housing can be roughened or chemically treated.

In a further embodiment, the housing 160 includes a non-bone penetrating tip or nose, made of a resorbable material. Prior to implanting the marker, a hole is drilled in the bone using a guide tube and then the implantable marker is guided towards the pre-drilled hole by the guide tube and the tip self locates the implantable marker in the pre-drilled hole and then the implantable marker is screwed into the pre-drilled hole so as to drive the implantable marker into the bone.

With reference to FIG. 5 there is shown a system 200 according to an embodiment of the invention, including wireless tracking subsystem 130, computer control system 122 and an image system 202 in the form of an X-ray based imaging system. Imaging system 202 includes a first X-ray source 204 bearing a marker 206 trackable by the tracking system 130. Imaging system 202 also includes an X-ray detector 208 which can be in the form of an X-ray cassette including an X-ray sensitive film located at an imaging plane of the X-ray cassette. X-ray cassette 208 also bears a marker 210 detectable and trackable by the tracking system 130. The X-ray image marker 210 has a known positional relationship with the position and orientation of the imaging plane of the X-ray detector 208.

In the embodiment in which an X-ray cassette is used, the developed X-ray film can be scanned and digitised to provide the X-ray image data in a digital form. In an alternate embodiment, the X-ray detector 208 is a digital X-ray detector which generates X-ray image data directly and again the position of the X-ray image marker 310 relative to the position and orientation of the imaging plane of the digital X-ray image detector is known. There is a known positional relationship between the imaging plane of the X-ray detector 208 and the X-ray source 204.

A patient 214 having a first marker 217 detectable by the tracking system and implanted in the femur is positioned adjacent the X-ray detector. The patient 214 also has a second marker 218 implanted detectable by the tracking system implanted in their tibia. In other embodiments, only a single bone marker is implanted in a one of the patient's bones. In other embodiments, more than one marker can be implanted in each bone and more than two bones can have a one or more markers implanted therein. As indicated above, other tracking technologies can be used and the invention is not limited to the use of markers wirelessly detectable radio frequencies. For example, bone markers 216, 218 can be attached to the bone using a support structure or can be attached to the patient's body above the bone rather than within the bone.

Each of the bone markers, 218, 216 is displaced by a distance d, in a direction substantially perpendicular to the plane of the imaging plane of the X-ray detector 208. The distance d is preferably relatively small, e.g. a few cms and preferably in the range between approximately 10-30 cm. Distance “d” is required for each image to calculate linear magnification of the X-ray image (which will later be displayed on the computer screen for navigation) with respect to the actual size of the patients bone. The same magnification factor is also applied to the navigated instruments and/or implants and their tracked movements during navigated surgery.

A method allowing images of a bone to be registered with the position of the bone will now be described with further reference to FIG. 6. FIG. 6 shows a flowchart illustrating a computer implemented method 250 implemented by a computer program executed by computer control system 122. At step 252, the program code is called and initiates. At step 254, the position of the bone implanted markers 216, 218 and of the X-ray image marker 210 in the reference frame of the tracking system are captured and stored. The computer program also determines the separation d between the imaging plane of the X-ray detector 208 and each of the bone implanted markers 218 and 216. Using d a linear magnification factor for the patients bones is calculated and stored for future use. The position of each of the bone implanted markers in the reference frame of the tracking system as is the position of the X-ray image marker 210 and the computer 122 also has access to the relative positioned orientation of the imaging plane relative to the position and orientation of the X-ray image marker 210.

In one embodiment, in order to allow a 3D image of the bone to be displayed, X-ray images of the bone from at least two different directions are captured. This can be achieved in a number of ways. One way of achieving this is using a single X-ray source and having the patient change the position of their bones, e.g. by rotating to approximately 90°. Another method would be to move the position of the X-ray sources, as illustrated by arrow 220 in FIG. 5. The direction of arrow 220 is schematic only and in practice, the X-ray source would be rotated through approximately 90° around the bone or bones of interest. In a further embodiment, a second X-ray source 204 which bears a further marker 206 detectable by the tracking system 130. The first X-ray source 204 and second X-ray source 204′ are preferably positioned to capture images at approximately 90° of each other. Method 250 will be described below using the example of a single X-ray source which is moved.

At step 256, with the X-ray source in a first position, an image of the patient's bones is captured and the position of the X-ray source in the reference frame of the tracking system is determined and captured. The captured image is correct using the linear magnification factor calculated previously and the corrected image is used subsequently. Then at step 258, the X-ray source is rotated through approximately 90° about the patient's body and a second image of the relevant body part is captured and again the position of the X-ray source in the reference frame of the tracking system is determining captured. The captured second image is correct using the linear magnification factor calculated previously and again the corrected second image is used subsequently. As there is a known relationship between the position of the X-ray source and the imaging plane of the X-ray detector, with the X-ray source in the first position, and as the position of the X-ray camera in the first position and in the second position in the reference frame of the tracking system are obtained from the track positions of the markers, it is possible to determine the relationship between the first captured X-ray image and the second captured X-ray image. Hence when a one of the images is registered with the bone position, the second image can also be registered or vice versa, as their fixed position relationship is known.

Method 250 is modified slightly in other embodiments of the imaging system 200. If the patient 214 moves, rather than the camera, then it is not necessary to track the position of the camera as there is a known fixed spacial relationship between the position and orientation of X-ray camera 204 and detector 208. Instead, the program uses the change in the orientation and position of the implanted markers (assuming the bone is otherwise in the same position) to determine the positional relationship between the first and second X-ray images. In the embodiment in which two separate X-ray camera sources 204, 204′ are used, either the position of the first and second X-ray cameras can be detected or alternatively, there can be a known positional relationship between each of the cameras and the X-ray detector 208 which can be used to derive the positional relationship between the first and second X-ray images.

After the two X-ray images have been captured, irrespective of the method used, at step 260, a one of the X-ray images is brought into registration, in the reference frame of the computer control system, with the actual position of the bone in the reference frame of the computer control system. The method of registering the bone image and bone position will be described in greater detail below. Steps 254, 256 and 258 correspond generally to step 106 of method 100 and step 260 corresponds generally to step 108 of method 100. Step 260 does not necessarily include the generation of a registered image, but merely includes determining the relevant data to allow an image (either one of the captured images or an image derived from one of the captured images) to be mapped on to the position of the bone in the reference frame of the tracking system.

With reference to FIG. 7 there is shown an operating room or operating theatre environment 270 and with reference to FIG. 8 there is shown a method of using the registered image 290 corresponding generally to steps 110, 120 and 122 of method 100.

The operating room environment 270 includes the patient 214 and a patient support 272, e.g. an operating table. A tracking system 132 is provided with the patient located with the surgical site and trackable markers 216, 218 located generally within the tracking system magnetic field working volume 138. Also shown is computer control system 122 and a component 274 bearing a marker 276 detectable and trackable by the tracking system 132. The component 274 can be any component or device used in the surgical procedure, such as a surgical tool or instrument or an orthopaedic implant. Also shown is a visual display 277 including the registered image 278 on the display of computer control system 122. Computer control system 122 and tracking system 132 can be the same computer control system and tracking system used to capture the positional data previously, in which case the tracking system and computer control system are provided as a portable system. Or alternatively, they can be merely similar systems having the same tracking functionality but slightly different computer implemented control functionality.

In an embodiment in which the computer control system and tracking system are portable and the same as those used previously, then the computer control system 122 already has access to the data required to generate the registered image and can have had the digitised X-ray image data downloaded or stored therein. In an alternate embodiment, the data required to register the image and the image data can be supplied to computer system 122 on some form of computer readable medium or transmitted thereto over a network, such as a local area network or wide area network and including both wired and wireless networks.

FIG. 8 shows a flowchart illustrating a method for carrying out a computer aided surgical procedure 290 utilising the registered image. The method begins at step 292 with the patient located in the operating room on the operating table with their knee joint within the tracking volume 138 of the tracking system 132. At step 294, computer system 122 makes the necessary calculations to map the bone image data on to the position of the bone in the reference frame of the tracking system.

After step 272, at step 298, the position of the implanted bone markers, 216, 218 is detected and the positions and orientations of the bone markers 216 and 218 are determined. Using the detected positions and orientations of bone markers 216, 218, at step 300 a three dimensional image of the patient's bones is rendered from the captured X-ray image data and the three dimensional image 278 is graphically displayed. As the image data is already registered with the positions of the actual body parts and as the position and orientations of the body parts has been determined in the operating room, it is possible to display a registered image of the bones without requiring any further registration procedures in the operating room. Also, as the orientations of the markers 216, 218 has been detected, it is possible to render, from the X-ray image data the appropriate three dimensional view of the bone from the direction corresponding to the orientation of the bones in the operating room.

After the registered image has been displayed at step 300, at step 302, the tracking system can detect the presence of marked tools, instruments and implants. Using the unique marker identifiers transmitted by the markers, the computer system 122 looks up graphics data and images associated with the marker identifiers. Some of the images to be displayed are corrected using the magnification factor determined previously to ensure that the representation of the tools and instruments are the appropriate size for the size of the bone images. After applying the magnification correction for each image for tools to be displayed and for navigation, at step 304, a graphical representation 180 of the physical component 274 is displayed in display 277 with the graphical representation being located at the position of the component and with the correct orientation of the component within the reference frame of the tracking system and relative to the position of the body part. Hence at step 304, a display 277 is provided showing a graphical representation of any tools, instruments and implants relative to the position of the registered bone image. At step 306, the surgical procedure can be started and carried out.

It will be appreciated that no invasive surgical steps have yet been carried out, but that already a registered image of the body part is available to the surgeon. For example step 306 can include planning the surgical procedure and then carrying out the surgical procedure using the navigated instruments and implants and using the displayed images 277 to guide the surgeon. At step 308, the positions of the markers within the working volume 138 is constantly tracked and if it is determined that the procedure is still ongoing and that tracking is still active at step 310, then the display is constantly updated, as represented by line 312 and a real time display of the current positions of the bone and instruments, tools and implants is rendered at step 304. When it is determined that the surgical procedure has been completed then the method can end at step 314.

With reference to FIG. 9 there is shown a further embodiment of a system 320 according to the invention including control computer 122, tracking system 132 and patient 214 having bone implanted markers 216, 218. In this embodiment, the imaging system is a CT scan system 322 including an image acquisition part 324 including an imaging plane 326. A patient support 328 is also included in the form of a table on a stand 330. A computer control system 322 generally controls the CT scan apparatus and stores the CT scan data. Computer control system 322 can control the position of table 328 including changing its height, as illustrated by arrow 332 and controlling the position of the patient within the image acquisition part 324, as illustrated by arrow 334 so as to collect a sequence of images of “slices” through the patient's bones.

There is a positional relationship between the imaging plane 326 and the position of table 328. FIG. 10 illustrates a first method of using a first embodiment of system 320 in order to allow a registered image to be generated and FIG. 11 illustrates a second method for using a second embodiment of system 320 to allow registered images to be generated.

In a first embodiment of system 320, table 328 includes a marker 336 detectable and trackable by the tracking system 132. The marker 336 is located at a known position relative to the table 328 and, as there is a known relationship between the position of the table and the imaging plane 326 at any table position, the position of the imaging plane 326 in the reference frame of the tracking system can be determined. The patient 214 is positioned on the table 328 and does not move during the CT scan procedure.

FIG. 10 show a flowchart illustrating data processing operations carried out by computer control system 122 and implemented by a computer program 350. Program 350 is called and initiates at step 352. At step 354, the computer control system 122 determines the positions of markers 218, 216 and 336 in the reference frame of the tracking system. Computer 122 also determines the current positional relationship between table 328 and imaging plane 326 corresponding to the detected marker position 336. At step 356, the CT scan of the patient's knee is carried out. At step 358, the CT scan image data is downloaded from CT scan system control 332 together with data indicating the position of table 328 for each captured CT scan image. Then at step 360, at least a one of the scans of the patient's body part is registered with the position of the body part in the reference frame of the tracking system 132.

As there is a fixed spacial relationship between all of the images, once a one of the images has been brought into registration, the entire CT scan set of images can become registered. The bone markers 218, 216 indicate the position of the actual body part in the reference frame of the tracking system and using the position of table 328 in the reference frame of the tracking system, determined using the detected position of marker 336 and knowing the positional relationship between table 328 and image plane 326, the scanned images can be brought into registration with the bone positions as will be described in greater detail below.

FIG. 11 shows a flowchart illustrating a computer implanted method allowing registered images to be generated using a second embodiment of system 320 and implemented by computer program 370. In this method, the table 328 of CT system 322 does not include marker 336. Rather, this method is based on capturing at least one image of the bone which includes an image or at least a part of the marker in the bone and determining the position of the marker at the time that the image is captured.

The table 328 is driven until a one of bone markers 218, 216 is located near the imaging plane 326. At step 374, the position of markers 216 and 218 is continuously determined and captured from tracking system 132 as, at step 376, the CT scan is carried out and the position of the markers 218, 216 is determined and captured for each of the scan images collected. It is preferred to capture a plurality of scan images including both the borne and marker although it is only necessary to capture at least one image showing both the bone and at least a part of one of the markers.

At step 378, the CT scan image data is downloaded from the CT system controller 332 to control computer 122 together with data indicating the position of the table. The position of the table is correlated with the marker positions such that each marker position is correlated with a corresponding CT scan image.

Bone markers 216, 218 determine the position and orientation of the bone in the reference frame of the tracking system. At least a one of the CT scan images also shows at least a part of one of the markers and therefore the position of that image in the reference frame of the tracking system is known. Then at step 380, the CT image including the markers is brought into registration with the position of the bone in the reference frame of the tracking system thereby registering the entire set of CT scan images. It is not necessary to actually register the images at step 380 but merely to determine the mapping vector or translation required in order to allow the registered image to be generated subsequently. Hence at step 380 and similarly at step 360, all that is determined is the mapping that determines the relationship between the position of the captured images in the reference frame of the scanning system and the bone position in the reference frame of the tracking system.

With reference to FIG. 12 there is shown a flowchart illustrating a method 390 in which a registered image obtained from the system 320 shown in FIG. 9 can be used. Method 390 is generally similar to method 290 as shown in FIG. 8 and so will not be described in great detail. In method 390, instead of displaying X-ray images, CT scan images are displayed and again the displayed CT scan images can either be the actual images captured or are preferably three dimensional images derived from the captured CT scan images which reflect the appearance of the bone with the patient in the orientation on the operating room table as determined by the position and orientation captured from the bone markers.

With reference to FIG. 13, there is shown a schematic representation of a software architecture 420 for the computer control system 122. Software architecture 420 includes a number of conceptual parts which in practice can be implemented by separate programs or a single program with different parts interacting with each other to provide the desired functionality. Architecture 420 should be understood at a conceptual level and is not intended to limit the invention, e.g. to three separate programs only.

Computer control system 122 includes an operating system under which various application programs forming part of architecture 420 execute. A tracking program or programs 422 processes signals and/or data received from the tracking system 132 and provides marker IDs, positions and orientation data to a registration program 424 and/or to a computer aided surgery program or programs 426. Where different computer control system 122 and tracking systems 132 are used for the image registration and surgical procedure, then the image registration system uses tracking program 432 and registration program 424 only. The computer aided surgery module then uses tracking module 422 and computer aided surgery module 426. When a single computer control system 122 ands tracking system 132 are used to implement both the image registration and computer aided surgery procedures, then the tracking, registration and computer aided surgery modules are all provided.

As illustrated in FIG. 13, a database 428 for storing data relating to and derived from the trackable markers can be provided. Database 428 can store various data items, including patient identified data items, marker ID data items, which uniquely identify each marker. Other data associated with the markers can also be stored in marker database 428, including the position of a marker relative to a part of an imaging system, such as the position of marker 210 relative to the imaging plane and marker 336 relative to the table. Also, the implant, instrument or tool associated with a marker can also be identified from database 428. A database 430 is also provided which stores the “raw” X-ray or CT scan data and which is used in rendering the three dimensional images of the body parts for display during the computer aided surgical procedure. Other data associated with the body scan images can also be stored in database 430, including the relative position of a scan to another scan so as to allow multiple images to be registered concurrently once a one of the images has been registered.

The functioning of registration module 424 will be described in greater detail with reference to FIGS. 14 and 15 below. Computer aided surgery module 426 includes a number of applications, including applications providing surgical planning functionality, instrument, tool and implant navigational functionality and image guided surgery functionality. The computer aided surgery module 426 interacts with tracking module 422 to obtain marker IDs and current marker position and orientation data, as well as bone position and orientation data so as to allow the real time display of a representation of the registered image and representations of any tools and/or implants as illustrated in FIG. 7 and corresponding generally to method steps 304 and 402.

Registration program 424 can have access both to the marker database 428 and scan database 430 and can either store data required for generating a registered image in the scan database or supplied to the computer aided surgery module 426 so as to allow the registered image to be generated. Registration program 424 includes instructions for carrying out methods 250, 350 and 370.

With reference to FIGS. 14 and 15, the processes involved in registering the image data with the position of the body part (corresponding generally to method steps 260, 360 and 380) will now be described with reference to FIGS. 14 and 15. FIG. 14 shows a flowchart illustrating a method for allowing a registered image to be generated implemented by computer program 440. The registration process 440 is called and begins at step 442. At step 444, the position of at least one bone marker is determined in the reference frame of the tracking system. FIG. 15 provides a graphical representation of the operations carried out by registration process 440. In FIG. 15, a graphical representation of the position of bone marker 216, position 454 and of bone marker 318, position 456, in the reference frame of the tracking system 452 is determined. At step 446, the position of the image 460 in the reference frame 458 of the tracking system is determined. Then at step 448, a vector 461 in the reference frame of the tracking system which maps the bone image 460 on to the position of the bone, as determined by the marker positions 554, 556 is determined and can subsequently be used to generate the registered image 464 which completes the registration procedure at step 450. In practice, vector 461 can be provided by a transformation matrix which is applied to the image data.

The mapping 461 which is required can be determined in a number of ways, depending on the system used. For example when marker 336 is used in CT scan table 328, then point 468 corresponds to the detected position of marker 336 in the reference frame of the tracking system and position 466 corresponds to the position of the marker relative to the image. The mapping vector 461 is therefore the vector required to map position 466 on to position 468 in the reference frame of the tracking system. In the embodiment in which image 460 also includes an image of a part of the marker, then point 466 corresponds to the position of the marker in the image and the appropriate mapping is that vector which maps the position of the marker 466 in the image on to the detected position of the marker 468 in the reference frame of the tracking system at the time that the image 460 was captured.

In the X-ray system embodiment 200 point 469 corresponds to the position of bone implant 216 in the imaging plane of the X-ray detector and point 470 corresponds to the position of bone implant 218 in the imaging plane of the X-ray detector. Point 454 corresponds to the position of bone marker 216 and point 456 corresponds to the position of bone marker 218 in the reference frame of the tracking system. Using marker 210, the position of the image 460 in the reference frame of the tracking system is known and so the positions of the bone markers relative to the image, 469, 470 are derivable therefrom. Therefore step 448 corresponds to determining the vector required to map points 469 and 470 in image 460 on to the detected bone positions 454 and 456 so as to generate the registered image 464.

In another embodiment, the bone markers 216, 218 are not imaged in the x-ray (so not seen on 458 as 469+470) but are located in the same bone as is shown in the image. The image can still be registered as the position of the markers and hence the bone is registered in the computer control system even though not shown in the image, and as there is a known fixed positional relationship between the markers and the parts of bone shown in the image.

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

FIG. 16 illustrates a typical computer system that, when appropriately configured or designed, can serve as an image analysis apparatus of this invention. The computer system 500 includes any number of processors 502 (also referred to as central processing units, or CPUs) that are coupled to storage devices including primary storage 506 (typically a random access memory, or RAM), primary storage 504 (typically a read only memory, or ROM). CPU 502 may be of various types including microcontrollers and microprocessors such as programmable devices (e.g., CPLDs and FPGAs) and unprogrammable devices such as gate array ASICs or general purpose microprocessors. As is well known in the art, primary storage 504 acts to transfer data and instructions uni-directionally to the CPU and primary storage 506 is used typically to transfer data and instructions in a bi-directional manner. Both of these primary storage devices may include any suitable computer-readable media such as those described above. A mass storage device 508 is also coupled bi-directionally to CPU 502 and provides additional data storage capacity and may include any of the computer-readable media described above. Mass storage device 508 may be used to store programs, data and the like and is typically a secondary storage medium such as a hard disk. It will be appreciated that the information retained within the mass storage device 508, may, in appropriate cases, be incorporated in standard fashion as part of primary storage 506 as virtual memory. A specific mass storage device such as a CD-ROM 514 may also pass data uni-directionally to the CPU.

CPU 502 is also coupled to an interface 510 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 502 optionally may be coupled to an external device such as a database or a computer or telecommunications network using an external connection as shown generally at 512. With such a connection, it is contemplated that the CPU might receive information from the network, or might output information to the network in the course of performing the method steps described herein.

Although the above has generally described the present invention according to specific processes and apparatus, the present invention has a much broader range of applicability. In particular, aspects of the present invention is not limited to any particular kind of orthopaedic performance and can be applied to virtually any joint or body structure, whether including an implant or implants or not, with relatively moving bones where an image of the body part in a form in which it can be registered is desired. Thus, in some embodiments, the techniques of the present invention could provide a registered image for use surgery not involving implants, e.g. a cruciate ligament operation, as well as implant replated surgical techniques. One of ordinary skill in the art would recognize other variants, modifications and alternatives in light of the foregoing discussion.

It will also be appreciated that the invention is not limited to the specific combinations of structural features, data processing operations, data structures or sequences of method steps described and that, unless the context requires otherwise, the foregoing can be altered, varied and modified. For example different combinations of structural features can be used and features described with reference to one embodiment can be combined with other features described with reference to other embodiments. Similarly the sequence of the methods step can be altered and various actions can be combined into a single method step and some methods steps can be carried out as a plurality of individual steps. Also some of the structures are schematically illustrated separately, or as comprising particular combinations of features, for the sake of clarity of explanation only and various of the structures can be combined or integrated together or different features assigned to other structures.

1. A method for generating a registered image of a body part of a patient for use in a computer aided surgical procedure, the method comprising:
attaching a marker detectable by a tracking system to the body part prior to any surgical steps of the surgical procedure, the tracking system having a reference frame;
detecting the position of the marker in the reference frame;
capturing at least a first image of the body part using an imaging system;
obtaining an indication of the position of the first image relative to the reference frame of the tracking system; and
determining a mapping to bring the first image into registration with the position of the body part.

2. The method of claim 1, and further comprising the step of mapping the first image into registration with the position of the body part in the reference frame of the tracking system.

3. The method of claim 1, wherein the step of obtaining an indication of the position of the at least first image relative to the reference frame of the tracking system includes the step of detecting the position in the reference frame of the tracking system of a further marker attached to a part of the imaging system using the tracking system.

4. The method of claim 1, wherein the first image includes the marker and at least a part of the body part, and wherein the position of the marker is detected when the first image is captured thereby providing the indication.

5. The method of claim 2, and further comprising the step of displaying the registered image during the computer aided surgical procedure.

6. The method of claim 5, wherein the surgical procedure is an orthopaedic procedure.

7. The method of claim 1, wherein the step of attaching the marker includes implanting the marker in a bone of the patient.

8. The method of claim 7, wherein the step of implanting the marker includes percutaneously implanting the marker.

9. The method of claim 1, wherein the marker is wirelessly detectable at radio frequencies by the tracking system.

10. The method of claim 1, wherein the imaging system is an X-ray system.

11. The method of claim 11, wherein the position of the marker is detected with the patient standing.

12. The method of claim 1, wherein the marker is wirelessly tracked using a magnetic tracking system.

13. The method of claim 10, wherein the step of obtaining an indication of the position of the at least first image relative to the reference frame of the tracking system includes the step of determining the position of an X-ray detector in the reference frame of the tracking system.

14. The method of claim 10, and further comprising the step of capturing a second image of the body part using the X-ray system, and wherein the second image is in a second direction different to a first direction in which the first image was captured.

15. The method of claim 14, wherein the step of capturing the second image includes moving the patient relative to the X-ray system.

16. The method of claim 14, wherein the step of capturing a second image includes moving an X-ray source relative to the patient, and further comprising the step of determining the position of the X-ray source in the reference frame of the tracking system when the second image is captured.

17. The method of claim 14, wherein the first image is captured using a first X-ray source and wherein the step of capturing the second image includes using a second X-ray source at a second position which is different to a first position of the first X-ray source.

18. The method of claim 14, further comprising the step of generating a three dimensional image of the body part from the first and second images.

19. The method of claim 10, further comprising the step of determining the distance between the body part and an imaging plane of an X-ray detector along a direction perpendicular to the plane of the imaging plan and using the distance to correct the first image captured by the X-ray detector.

20. The method of claim 1, wherein the imaging system is a CT scan or an MR scan system.

21. The method of claim 20, wherein the body part of the patient is located on a patient support part of the imaging system when the first image is captured and further comprising determining the position of the patient support part in the reference frame of the tracking system.

22. The method of claim 21, further comprising the step of mounting a marker detectable by the tracking system on the patient support part.

23. The method of claim 20, wherein the body part of the patient is located on a patient support part of the imaging system when the first image is captured, and further comprising the step of determining the position of an imaging plane of the scan system relative to the position of the patient support part.

24. The method of claim 20, wherein the first image includes the marker and at least a part of the body part and wherein the position of the marker is detected when the first image is captured.

25. The method of any preceding claim and further comprising the steps of:
attaching a further marker detectable by the tracking system to a further body part prior to any surgical steps of the image guided surgical procedure; and
detecting the position of the further marker in the tracking reference frame.

26. The method of claim 25, and wherein the step of mapping the first image into registration with the position of the body part includes using the position of the further marker.

27-50. (canceled)
